(19) United States
(12) Patent Application Publication
(10) Pub. No.: US 2024/0158344 A1
(43) Pub. Date: May 16, 2024

(54) PROCESS FOR ISOLATION OF CAPSANTHIN AND OTHER CAROTENOIDS FROM PAPRIKA OLEORESIN

(71) Applicant: KEMIN INDUSTRIES, INC., DES MOINES, IA (US)

(72) Inventor: Frederick Khachik, Urbandale, IA (US)

(73) Assignee: KEMIN INDUSTRIES, INC., DES MOINES, IA (US)

(21) Appl. No.: 18/381,372

(22) Filed: Oct. 18, 2023

Related U.S. Application Data
(60) Provisional application No. 63/424,514, filed on Nov. 11, 2022.

(51) Int. Cl. C07C 403/24

(52) U.S. Cl. CPC C07C 403/24

(57) ABSTRACT

The present invention relates to novel processes for isolation of capsanthin and several other carotenoids from paprika oleoresin that is a concentrated extract of paprika fruit (Capsicum annuum). In another aspect, the present invention relates to the use of mild non-aqueous and aqueous saponification processes for isolation of high purity capsanthin and other carotenoids from paprika oleoresin at temperature between about 20 to 50° C. In another aspect, the present invention relates to saponification processes resulting in a crystalline mixture of capsanthin, β-carotene, β-cryptoxanthin, and zeaxanthin that can be separated by solvent extraction and crystallization to provide high purity capsanthin, as well as other carotenoids.

### CROSS-REFERENCE TO RELATED APPLICATIONS

The present application claims the benefit of priority to U.S. Provisional Patent Application No. 63/424,514, filed Nov. 11, 2022, entitled “PROCESS FOR ISOLATION OF CAPSANTHIN AND OTHER CAROTENOIDS FROM PAPRIKA OLEORESIN,” the entire disclosure of which is incorporated herein by reference in its entirety.

### INTRODUCTION

The present invention relates to novel processes for isolation of capsanthin and several other carotenoids from paprika oleoresin that is a concentrated extract of paprika fruit (Capsicum annuum). In another aspect, the present invention relates to mild saponification processes for the isolation of high purity capsanthin and other carotenoids, such as from paprika oleoresin at lower temperatures, such as between ambient to about 48° C. In another aspect, the present invention relates to processes for obtaining capsanthin, zeaxanthin, β-carotene, and β-cryptoxanthin from paprika oleoresin by saponification of carotenoid esters, followed by crystallization and separation of these carotenoids.

### BACKGROUND OF THE INVENTION

The present invention relates to novel processes for isolation of capsanthin and several other carotenoids from paprika oleoresin that is a concentrated extract of paprika fruit (Capsicum annuum). In another aspect, the present invention relates to mild saponification processes for the isolation of high purity capsanthin and other carotenoids, such as from paprika oleoresin at lower temperatures, such as between ambient to about 48° C. In another aspect, the present invention relates to processes for obtaining of capsanthin, zeaxanthin, β-carotene, and β-cryptoxanthin from paprika oleoresin by saponification of carotenoid esters, followed by crystallization and separation of these carotenoids.

Capsanthin is the major carotenoid in paprika oleoresin that is esterified with fatty acid esters. In addition to capsanthin, other major carotenoids in paprika oleoresin are: β-carotene, β-cryptoxanthin, and zeaxanthin. Because capsanthin undergoes degradation in hot alkaline solution, conventional saponification of this esterified carotenoid in paprika oleoresin at temperature above 50° C. results in significant loss of this heat-labile carotenoid. Accordingly, there has been a long-felt need for a saponification process that does not result in significant loss of these compounds.

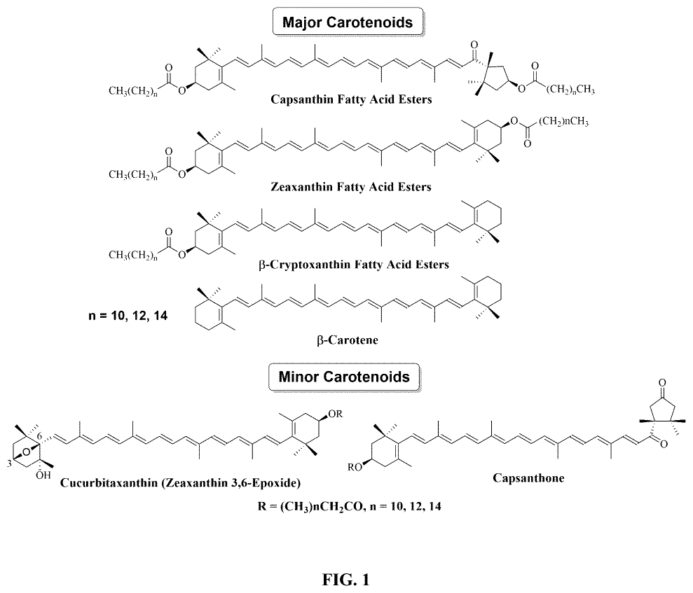

Paprika oleoresin is an extract of paprika fruit (Capsicum annuum) with approximately 5% major carotenoids and about 4% minor carotenoids; the major carotenoids are consisted of mainly trans- and cis-isomers of capsanthin (40-48%), β-carotene (11%-14%), β-cryptoxanthin (9%-10%), zeaxanthin (10%-12%), cucurbitaxanthin (5-7%), and capsanthone (5-6%). Except β-carotene, these carotenoids are esterified with palmitic, myristic, and lauric acids. Therefore, the concentrated extracts from paprika fruit (paprika oleoresin) are saponified to transform capsanthin fatty acid esters to unesterified free capsanthin. Similarly, upon saponification, zeaxanthin and β-cryptoxanthin fatty acid esters are converted to their corresponding hydroxycarotenoids. The chemical structures of major paprika carotenoids are shown in FIG. 1. The inventor has also identified a number of minor carotenoids in paprika oleoresin; these are: zeaxanthin 3,6-epoxide (cucurbitaxanthin); capsanthone, zeaxanthin 5,8-furanoxide (mutatoxanthin); capsanthin 3,6-epoxide; capsanthin 5,8-furanoxide (capsochrome), and karpoxanthin.

In addition to the above carotenoids, paprika fruit also contains capsorubin, cryptocapsin, karpoxanthin, and their corresponding epoxides [J. Deli, P. Molnar, Current Organic chemistry. (2002), 6(13), 1197-1219]. Eleven minor apocarotenoids have also been isolated and identified in fruits of the red paprika (Capsicum annuum) [Maoka et al. J. Agric. Food Chem. (2001), 49, 1601-1606]. Due to the complex nature of carotenoids in paprika oleoresin, the focus of the present invention is on the major carotenoids mentioned above.

Over the past several decades, the health benefits of dietary carotenoids such as lutein, zeaxanthin, β-cryptoxanthin, and β-carotene have been well-established. Similarly, the biological activity of capsanthin in disease prevention has been the subject of intense studies. In 1998, Matsufuji et al. (J. of Agric. & Food Chem. 46(9), 3468-3472) reported on antioxidant activity of capsanthin and its fatty acid esters by measuring the free radical oxidation of methyl linoleate. The antioxidant, antinociceptive, and anti-inflammatory effects of carotenoids extracted from dried pepper (Capsicum annuum L.) has also been documented by Hernandez-Ortega et al. [J. of Biomed. and Biotech. (2012), 524019, 10 pp]. In another study, Narisawa et al. [Proceedings of the Society for Experimental Biology and Medicine, 2000, 224(2), 116-122] reported on the prevention of colon carcinogenesis in rat by capsanthin and capsanthin-rich paprika juice. Similarly, cancer chemopreventive activity of carotenoids in the fruits of red paprika (Capsicum annuum L.) was demonstrated by Maoka et al. [Cancer Letters. (2001), 172(2), 103-109]. Capsanthin has also been shown to inhibit both adipogenesis in 3T3-L1 preadipocytes obesity-induced inflammation and to prevent weight gain in high fat diet-induced obese mice [Jo, Sung Jun, Biomolecules & Therapeutics. (2017), 25(3), 329-336]. Further, dietary capsanthin has been shown to have an HDL-cholesterol-raising effect on plasma, and hepatic gene expression in rats [Aizawa & Inakuma, British J. of Nutrition. (2009), 102(12), 1760-1766]. Another important health benefit of capsanthin has been related to its ability in protecting against nonalcoholic fatty liver disease in mouse model [Joo et al. J. of Medicinal Food. (2021), 24(6), 635-644]. Capsanthin and paprika carotenoids also protect human dermal fibroblasts against UVB-induced DNA damage [Fernandez-Garcia et al. Photochem. & Photobiologic. Sci. (2016), 15(9), 1204-1211]. Finally, it has been reported that dietary paprika carotenoids that are absorbed into blood contribute to endurance performance of athletes by reducing oxygen (VO₂) and the heart rate [Maeda, Hayato; Nishino, Azusa; Maoka, Takashi, Advances in Experimental Medicine & Biology. (2021), 1261, 285-293].

Despite the important health benefits of paprika carotenoids, there are only a few patented processes on extraction and isolation of carotenoids from fruits of paprika. For instance, Duanmu et al. (CN101906254 A, 2010-12-08) reported on a method for extraction of capsanthin ointment from dry paprika by using dimethyl ether as solvent. However, this patent describes extraction and preparation of a concentrated extract of paprika containing capsanthin esters and does not subject the extract to saponification and isolation of purified capsanthin.

Reilly et al. (US Pub. No. 20110282083 A1 2011-11-17) described a process of converting esterified xanthophylls from Capsicum to non-esterified xanthophylls in 60-80% purities. Reilly et al. only focused on the isolation of zeaxanthin from paprika oleoresin, however. Moreover, the saponification process described in Reilly et al. employed hexane, methanol, and aqueous KOH or NaOH (45% solution) for saponification and isolation of zeaxanthin at temperatures ranging from 22-83° C. Because hydrolysis of esters in an aqueous basic solution does not proceed at an ambient temperature, it was necessary to carry out the saponification in this system at an elevated temperature. In some cases, Reilly et al. carried out the saponification of paprika oleoresin at an ambient temperature in methanolic KOH for isolation of zeaxanthin. However, the disclosure does not describe the isolation of purified capsanthin, and persons of ordinary skill in the art would understand that because of the use of aqueous base, the process requires the separation of organic and aqueous phases. In order to remove the aqueous phase, Reilly et al. employed decantation of the upper phase in a centrifuge bottle and washed the precipitate with methanol for isolation of zeaxanthin. Employing decantation in a centrifuge bottle is not a process that would translate to large-scale commercial production without the use of specialized equipment.

Jacob et al. (WO2011135519 A1, 2011-11-03) described a supercritical fluid extraction process to obtain deodorized paprika followed by saponification to obtain the Capsicum annuum extract as a water-dispersible powder. This process focused on the isolation of a capsanthin-rich carotenoid mixture from Capsicum annuum oleoresin and did not describe the isolation or purification of individual carotenoids, however.

Umigai et al. (JP2015174858 A, 2015-10-05) described a preparation of Capsicum annuum (red pepper) pigment extract containing β-cryptoxanthin (I) and capsanthin (II) at a ratio of a content of II to I≤3 that does not turn feces color red when ingested.

Sunilkumar et al. in U.S. Pat. No. 9,771,323 B2, Sep. 26, 2017, described the isolation and purification of β-cryptoxanthin from a plant source and a process for its preparation. The plant source was paprika oleoresin, but the authors employed a high temperature (80-85° C.) for 3-5 h for saponification followed by column chromatography for isolation of purified β-cryptoxanthin. Because capsanthin is sensitive to aqueous alkaline solutions at high temperature, the process described in U.S. Pat. No. 9,771,323 B2 is most definitely accompanied by significant losses of this carotenoid. It should be noted that U.S. Pat. No. 9,771,323 B2 did not describe a process for isolation of purified capsanthin. Details of the degradation of capsanthin in aqueous solutions at high temperature will be discussed in detail later in this application.

In another patented process, Sunilkumar et al. (U.S. Pat. No. 10,301,259 B2, May 8, 2019), described a process for preparation and composition of 0-cryptoxanthin from plant source for improving lung health, physical performance and cardio-respiratory fitness and reduction of oxidative stress markers. The plant source in this case was paprika oleoresin.

In another patented process, Deshpande et al. (U.S. Pat. No. 10,568,846 B2, Feb. 25, 2020), described a β-cryptoxanthin compositions, processes for preparation and uses thereof in which this carotenoid is isolated from paprika oleoresin according to the process of Sunilkumar et al. (U.S. Pat. No. 9,771,323 B2, Sep. 26, 2017) described earlier. Deshpande et al. focused on the use of a composition of 0-cryptoxanthin for improving lung health, physical performance and cardiorespiratory fitness. This patent described administering an effective amount a β-cryptoxanthin composition comprising an extract enriched with trans-β-cryptoxanthin to a subject undergoing exercise, wherein the extract comprises about 75 to 100% by weight of trans-β-cryptoxanthin, and wherein the amount is effective so as to reduce oxidative stress markers and increase antioxidant muscle enzymes in the subject undergoing exercise as compared to those of a subject that is undergoing exercise and has not been administered the β-cryptoxanthin composition.

Finally, recently a patent application has been published by Mehta (US 2022/0009886 A1, Jan. 18, 2022) that describes extraction of paprika carotenoids by methanol and supercritical fluid extraction followed by saponification with alcoholic KOH at high temperatures in the range of 75-80° C. This report also points out that carotenoids were purified by ethyl acetate using counter current extraction. However, the disclosure provided no detail or discussion related to saponification and purification of carotenoids.

As described in detail above, the published work to date that has focused on the isolation of β-cryptoxanthin and zeaxanthin from paprika oleoresin has consistently disclosed the use of high temperature during the saponification process that necessarily results in the degradation of these heat-labile target carotenoids, such as capsanthin. There has been an unmet need for a process that employs the mild conditions needed for saponification of carotenoid esters and the isolation of capsanthin and other carotenoids from paprika oleoresin.

For these and other reasons, there is a need for the present invention.

### SUMMARY OF THE INVENTION

The present invention relates generally to processes for isolating and purifying capsanthin and several other major carotenoids from paprika oleoresin. In certain embodiments, the present invention relates to processes for saponification of the major carotenoid esters of capsanthin, zeaxanthin, and β-cryptoxanthin from paprika oleoresin followed by crystallization and separation of these carotenoids.

The inventor has surprisingly discovered novel processes with conditions that are appropriate for alkaline and heat-sensitive molecules, such as ambient to low temperature saponification processes, followed by the separation of carotenoids by crystallization without the use of column chromatography. These conditions solve the technical challenges of conventional saponification processes which result in significant loss of these target carotenoids.

More specifically, the inventor has surprisingly found that use of a mixture of propylene glycol (PG) and ethanolic potassium hydroxide (KOH) solution for saponification of carotenoids in paprika oleoresin. In at least one embodiment, the mixture comprises about 15% to about 20% ethanolic potassium hydroxide (KOH) solution for saponification of carotenoids in paprika oleoresin. For instance, in certain embodiments, the saponification of carotenoids in paprika oleoresin is accomplished at ambient temperature using about 15% to about 20% ethanolic potassium hydroxide (KOH) solution in acetone or 5% ethanolic solution without acetone.

Saponification according to the present invention provides a crystalline mixture of capsanthin, β-carotene, β-cryptoxanthin, and zeaxanthin that are separated by hexane extraction and crystallization to provide high purity capsanthin as well as other carotenoids.

In certain embodiments, saponification of paprika oleoresin was accomplished with a non-aqueous solution of potassium hydroxide (KOH) in ethanol (EtOH, 15-20%, wt.:wt.) and propylene glycol (PG) at 45-50° C. within 3-4 h. It should be noted that paprika oleoresin also contains β-carotene that is a hydrocarbon carotenoid and does not require saponification. At the end of saponification, ethanol was distilled under reduced pressure at 40-50° C. and the saponified oleoresin in propylene glycol (PG) was diluted with water and the KOH was neutralized with aqueous acetic acid. Filtration of this oleoresin at 50-70° C. afforded a crystalline mixture of capsanthin, β-carotene, β-cryptoxanthin, and zeaxanthin that were washed with 50-70° C. water to remove the PG, and the water-soluble anthocyanins and flavonoids. After drying, the crystalline mixture of these carotenoids was subjected to solvent extraction and crystallization for isolation of capsanthin and other carotenoids.

In certain embodiments, carotenoid esters in paprika oleoresin in acetone were saponified at ambient temperature within about 24 hours, for instance within about 12 hours, or alternatively within about 6 hours, and in preferred embodiments within about 3 to 6 hours, employing a 40-60% aqueous solution of potassium hydroxide (KOH) in ethanol. At the end of saponification, the product was neutralized with an aqueous solution of acetic acid and after distillation and recovery of acetone and ethanol under reduced pressure at 40-50° C., the saponified oleoresin was diluted with water. Filtration of this aqueous oleoresin at 50-70° C. afforded a crystalline mixture of capsanthin, β-carotene, β-cryptoxanthin, and zeaxanthin that were washed with 50-70° C. water to remove the residual acetic acid and the water-soluble anthocyanins and flavonoids. The crystalline carotenoids were dried at 50-60° C. under high vacuum overnight.

In certain embodiments, acetone is optionally used as a co-solvent with the solution of KOH in ethanol (wt.:wt.) to facilitate stirring of the saponification mixture. However, in certain embodiments, for instance when saponification of carotenoid esters in paprika oleoresin was carried out with 5% solution of KOH in ethanol (wt.:wt.), there was no need for acetone as co-solvent.

In certain embodiments, the saponification of carotenoid esters in paprika oleoresin was carried out with a 5% solution of KOH in ethanol at ambient temperature. At the end of saponification, KOH was neutralized with an aqueous solution of acetic acid and this was followed by distillation and recovery of ethanol under reduced pressure. The saponified oleoresin was then treated with hot water (about 50-70° C.) and the crystallized carotenoids were collected by filtration. The crystalline carotenoids were washed with water (about 50-70° C.) to remove the residual acetic acid, water-soluble anthocyanins and flavonoids, and then dried at about 60° C. under high vacuum.

In certain embodiment, the simultaneous saponification and separation of carotenoid esters in paprika oleoresin was carried out with a mixture of hexane and ethanol with slow addition of an aqueous solution of KOH in water (40-62%, wt.:wt.) for 4-5 h at ambient temperature followed by stirring for about 24 hours. At the end of saponification, KOH was neutralized with an aqueous solution of acetic acid and the saponified mixture was stirred at ambient temperature for 24 hours; this resulted in crystallization of capsanthin and zeaxanthin while β-carotene and β-cryptoxanthin remained in solution. The crystalline mixture of capsanthin and zeaxanthin were simply removed by filtration and were sequentially washed with hot water (about 50-70° C.) and hexane to increase the purity of these carotenoids. The crystallized mixture of capsanthin and zeaxanthin were then dried at about 50-60° C. under high vacuum. The filtrate from this crystallization contained β-carotene, β-cryptoxanthin, and other minor paprika carotenoids as well as their cis-isomers. The mixture of hexane and ethanol in the filtrate were recovered by azeotropic distillation of these solvents under reduced pressure. The recovered mixture of hexane and ethanol could be recycled without separation since saponification of carotenoid esters in paprika oleoresin was carried out with the mixture of these solvents. However, the ratio of these solvent had to be adjusted.

In certain embodiments, the crystalline mixture of carotenoids from various saponification processes was subsequently extracted with hexane, at about 25 to about 60° C., that solubilized β-carotene and β-cryptoxanthin while capsanthin and zeaxanthin remained insoluble in this solvent and were removed by filtration at ambient temperature. Because capsanthin and zeaxanthin exhibited slightly different solubility behavior in aqueous acetone, the partial separation of these carotenoids was accomplished by extraction. Therefore, according to certain embodiments, the extraction of a mixture of capsanthin (76%) and zeaxanthin (24%) with an aqueous solution of acetone followed by filtration resulted in a crystalline mixture of capsanthin (85%) and zeaxanthin (15%) in 85-90% purity.

Similarly, extraction of a mixture of β-carotene and β-cryptoxanthin with alcohols such as ethanol, 1-propanol, and 2-propanol resulted in an alcohol soluble fraction that consisted of β-cryptoxanthin while β-carotene remained as insoluble crystals. Each isolated carotenoid was further purified by crystallization with an appropriate solvent.

### DETAILED DESCRIPTION OF THE DRAWINGS

FIG. 1 illustrates the structures of all possible fatty acid esters of major carotenoids, capsanthin, β-cryptoxanthin, zeaxanthin as well as minor carotenoids, cucurbitaxanthin and capsanthone in paprika oleoresin; β-carotene is a hydrocarbon carotenoid and as a result is not esterified.

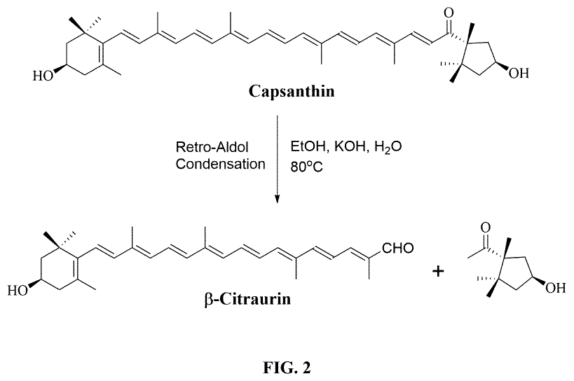

FIG. 2 illustrates degradation of capsanthin to β-citraurin by a retro-aldol condensation as published by Zechmeister and Cholnoky [Justus Liebigs Annalen der Chemie (1937), 530, 291-300].

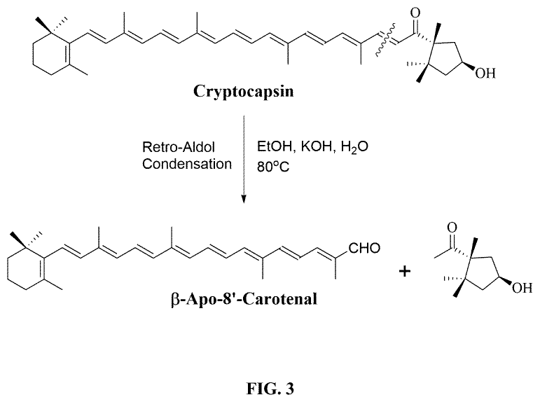

FIG. 3 Illustrates degradation of cryptocapsin to β-apo-8′-carotenal by a retro-aldol condensation as published by L. Cholnoky and J. Szabolcs (Tetrahedron Letters, No. 19, 1257-1259, 1963).

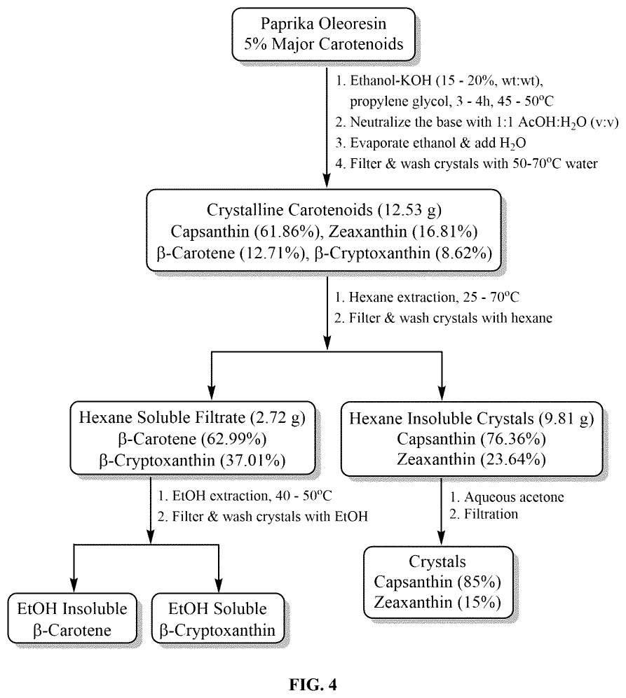

FIG. 4 is a flow chart for saponification of carotenoids in paprika oleoresin using ethanolic KOH in propylene glycol at 45-50° C. followed by separation of capsanthin, zeaxanthin, β-carotene, and β-cryptoxanthin by solvent extraction and crystallization.

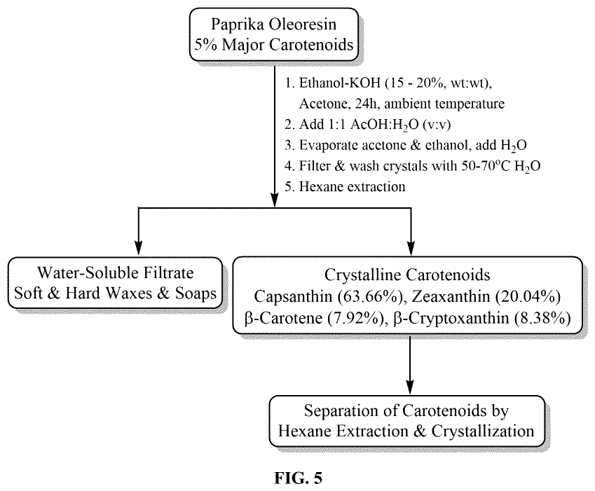

FIG. 5 is a flow chart for saponification of carotenoids in paprika oleoresin using ethanolic KOH (15-20%, wt.:wt.) in acetone at ambient temperature followed by separation of capsanthin, zeaxanthin, β-carotene, and β-cryptoxanthin by solvent extraction and crystallization.

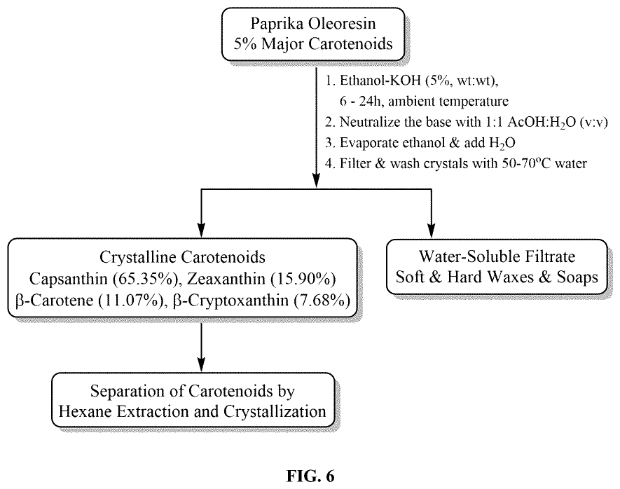

FIG. 6 is a flow chart for saponification of carotenoids in paprika oleoresin using ethanolic KOH (5%, wt.:wt.) at ambient temperature followed by separation of capsanthin, zeaxanthin, β-carotene, and β-cryptoxanthin by solvent extraction and crystallization.

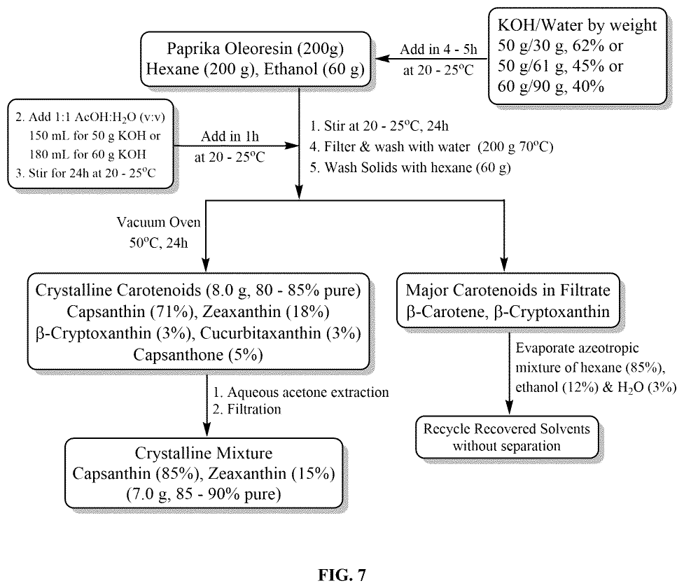

FIG. 7 is a flow chart for simultaneous saponification and separation of carotenoids in 200 g of paprika oleoresin in hexane and ethanol using aqueous solutions of KOH (40% or 45%, or 62%, wt.:wt.) at ambient temperature and crystallization of major carotenoids, capsanthin and zeaxanthin in high purity at the end of saponification.

### DETAILED DESCRIPTION OF THE INVENTION

The present invention relates to novel processes for saponification of carotenoid esters in paprika oleoresin under mild conditions to obtain a crystalline mixture of capsanthin, β-carotene, β-cryptoxanthin, and zeaxanthin which has the advantage of avoiding degradation of alkaline and heat-sensitive carotenoids, such as capsanthin. Another aspect of the present invention relates to novel processes for separation of these carotenoids and isolation of capsanthin in high purity by solvent extraction and crystallization.

Because conventional methods use alkaline solutions at a high temperature in order to drive the saponification of carotenoid esters in paprika oleoresin, it is imperative to describe the degradation of capsanthin under these conditions. In 1937, Zechmeister and Cholnoky [Justus Liebigs Annalen der Chemie (1937), 530, 291-300] demonstrated that capsanthin subjected to aqueous potassium hydroxide solution at 80° C. undergoes a retro-aldol condensation to β-citraurin as shown in FIG. 2. Similarly, hot aqueous potassium hydroxide treatment of cryptocapsin that is structurally similar to capsanthin was shown to result in cleavage of this ketocarotenoid to β-apo-8′-carotenal as shown in FIG. 3 (L. Cholnoky and J. Szabolcs, Tetrahedron Letters, No. 19, 1257-1259, 1963). Therefore, the present invention has developed ambient and low-temperature saponification processes for hydrolysis of capsanthin esters to avoid degradation of this carotenoid. As used herein, “low temperature” refers to about 20 to about 50° C. For instance, in certain embodiments, the saponification process occurs at ambient temperature (about 20-25° C.). In alternative embodiments, the saponification process occurs at temperatures ranging from about 45 to about 50° C. The resulting crystalline mixture of carotenoids obtained by these processes have been subjected to solvent extraction for separation of capsanthin and other carotenoids.

According to certain embodiments, the present invention relates to a process for low temperature saponification of carotenoid esters in paprika oleoresin to afford a mixture of capsanthin, β-carotene, β-cryptoxanthin, and zeaxanthin comprising:
treating paprika oleoresin with a non-aqueous solution of potassium hydroxide (KOH) or sodium hydroxide, or in alternative embodiments, other known alkali metal hydroxides, in ethanol (EtOH) or other C1-C3 alcohols or mixtures, and propylene glycol (PG) at a low temperature, for instance between about 45-50° C. to obtain a saponification mixture;
heating the mixture to a temperature, for instance to a range from about 40 to about 50° C. in order to saponify carotenoid esters;
treating the saponified paste with water and a 1:1 solution of acetic acid (AcOH) or other weak organic acids such as propanoic acid, butyric acid in water (v:v) to neutralize the base;
distilling and recovering ethanol under reduced pressure, for instance a range of about 200-120 torr at 45-50° C. to obtain a saponified paste;
treating the saponified paste with water to obtain a suspension of carotenoids;
filtering the suspension and washing the crystals to obtain a crystalline mixture of trans-capsanthin, trans-β-carotene, trans-β-cryptoxanthin, and trans-zeaxanthin; and drying the crystalline mixture, for instance in certain embodiments the crystalline mixtures is dried at high vacuum at 40-60° C., but in alternative embodiments the mixture is dried using other conventional drying techniques.

The process for low temperature saponification of carotenoid esters in paprika oleoresin using propylene glycol and ethanolic KOH followed by separation of capsanthin, β-carotene, β-cryptoxanthin, and zeaxanthin is shown in FIG. 4. According to at least one embodiment, the saponification of carotenoid esters in paprika oleoresin was accomplished with a non-aqueous solution of KOH (15-20%) in ethanol and propylene glycol (PG, 20% by weight of oleoresin) at 45-50° C. within 3-4 h. In one preferred embodiment, carotenoid esters in paprika oleoresin (50 g) were saponified with a 20% non-aqueous solution of KOH (10 g) in ethanol (40 g) and PG (10 g) at 45-50° C. within 3-4 h. After neutralizing the base with a 50% solution of acetic acid and water (23 mL, 1:1, v:v), ethanol was recovered by distillation under reduced pressure at 40-50° C. to prevent the loss of ethanol soluble carotenoids during filtration while propylene glycol (PG) remained in the saponified oleoresin due to its high boiling point (188.2° C.). Because carotenoids exhibit poor solubility in PG, the removal of this solvent prior to filtration was not necessary. In addition, the use of PG assisted filtration of saponified carotenoids. After ethanol evaporation, the saponified mixture was diluted with water (50 g) and the mixture was heated at 50-70° C. to obtain a suspension. The crystalline carotenoids were filtered and washed with 50 g of 50-70° C. water to remove the propylene glycol, residual acetic acid, and water-soluble anthocyanins and flavonoids. The wet crystalline mixture of carotenoids was dried at 40-60° C. under high vacuum to afford (4.80 g; 60% total carotenoids, 2.88 g) of capsanthin (61.86%), β-carotene (12.71%), β-cryptoxanthin (8.62%), and zeaxanthin (16.81%) that was subjected to solvent extraction for separation of individual carotenoids.

In another embodiment of the present invention, carotenoid esters in paprika oleoresin were saponified at ambient temperature with a non-aqueous solution of KOH in ethanol using acetone as co-solvent as depicted in the flow chart in FIG. 5. The weight ratio of acetone:oleoresin was in the range of 2:1 to 4:1 and the concentration of KOH in ethanol was in the range of about 10-20%. In a preferred embodiment of the present invention, for instance, carotenoid esters in paprika oleoresin (50 g) were solubilized in acetone (100 g) and the resulting mixture was saponified with a non-aqueous 20% solution of KOH (10 g) in ethanol (40 g) at ambient temperature within 24 hours. The use of acetone in the presence of KOH did not result in aldol condensation of acetone because saponification was carried out at ambient temperature. Under these conditions, significant amounts of soaps and waxes (23 g) were formed that were solubilized by addition of a 1:1 solution of acetic acid and water (20-24 mL). Acetone and ethanol were recovered by distillation under reduced pressure at 35-50° C. to afford a dark red paste. It should be noted that ethanol (b.p.=78.4° C.) and acetone (b.p.=56.5° C.) do not form an azeotropic mixture and can be readily separated and recovered by distillation due to the large difference in their boiling points. The saponified oleoresin was diluted with water (about 50 g). After heating the resulting mixture at 50-70° C., a homogeneous suspension was obtained. The crystalline carotenoids were filtered and washed with 100 g of 50-70° C. water to remove the residual acetic acid, and water-soluble anthocyanins and flavonoids. The wet crystalline mixture of carotenoids was dried at 40-60° C. under high vacuum to afford 4.17 g (60% total carotenoids, 2.50) of capsanthin, β-carotene, β-cryptoxanthin, and zeaxanthin that was subjected to solvent extraction for separation of individual carotenoids. This crystalline mixture was extracted with hexane to increase the purity of the mixture to 80-85% and to separate capsanthin and zeaxanthin from β-carotene and β-cryptoxanthin.

In another embodiment of the present invention, carotenoid esters in paprika oleoresin in acetone and ethanol were saponified with aqueous solution of KOH (40%) after 24 hours at ambient temperature that after work up and purification afforded a crystalline mixture of capsanthin (66.33%), zeaxanthin (20.63%), β-carotene (6.07%) and β-cryptoxanthin (6.97%) in 85% purity. Similarly, saponification of carotenoid esters in paprika oleoresin in acetone and ethanol with 45% aqueous solution of KOH after work up and purification afforded a crystalline mixture of capsanthin (66.11%), zeaxanthin (25.16%), β-carotene (2.81%) and β-cryptoxanthin (5.92%) in 83% purity.

In certain embodiments, where the solution of KOH in ethanol (wt.:wt.) was used for saponification of carotenoid esters in paprika oleoresin at ambient temperature, the use of acetone as co-solvent was necessary in order to facilitate stirring of the saponification mixture. However, in alternative embodiments of the present invention, carotenoid esters in paprika oleoresin were saponified with 5% solution of KOH in ethanol (wt.:wt.) without the use of acetone as co-solvent as depicted in the flow chart in FIG. 6. The weight ratio of ethanol:oleoresin was in the range of 3.80:1 to 5.70:1 and the weight ratio of oleoresin to KOH was in the range of 3.3:1 to 5:1.

In certain embodiments, carotenoid esters in paprika oleoresin (100 g) were saponified with a 5% non-aqueous solution of KOH (20 g) in ethanol (380 g) at ambient temperature within 24 hours. The saponified oleoresin was then treated with an aqueous solution of acetic acid (1:1, v:v) to neutralize the base and ethanol was recovered by distillation under reduced pressure. The saponified oleoresin was then treated with water (about 50-70° C.) and the crystallized carotenoids were collected by filtration and washed with water (about 50-70° C.) to remove the residual acetic acid, and water-soluble anthocyanins and flavonoids. After drying the crystals at 60° C. under high vacuum, the resulting crystalline mixture of carotenoids (9.00 g, 65% pure) was subjected to hexne extraction and crystallization for separation of capsanthin and other carotenoids as described earlier. According to certain embodiments, any drying method can be utilized.

Employing normal phase HPLC separation, the inventor has determined the detailed composition of the major paprika carotenoids after saponification. The relative composition of major carotenoids in paprika oleoresin after saponification at ambient or low temperature according to the present invention is shown in Table 1.

TABLE 1
The relative composition of major carotenoids after saponification of paprika oleoresin under various
conditions before crystallization determined by HPLC.^{a,b}

| | Paprika | Solvent | EtOH/KOH | Time, Temp. | Capsanthin | β-Carotene | β-Cryptoxanthin | Zeaxanthin |
|---|---|---|---|---|---|---|---|---|
| Exp. | Oleoresin, g | g | g/g, % wt:wt | h, ° C. | % trans, % cis | % | % | % |
| 1 | 50 | PG, 10 | 40/10, 20 | 3, 45-50 | 42.65, 12.30 | 19.21 | 13.37 | 12.47 |
| 2 | 50 | PG, 10 | 40/10, 20 | 4, 45-50 | 45.03, 13.60 | 17.34 | 12.73 | 11.30 |
| 3 | 100 | PG, 20 | 80/20, 20 | 4, 45-50 | 44.33, 15.28 | 16.37 | 12.50 | 11.52 |
| 4 | 100 | PG, 50 | 80/25, 16 | 4, 45-50 | 44.66, 17.91 | 13.96 | 10.88 | 12.59 |
| 5 | 100 | PG, 50 | 80/25, 16 | 4, 45-50 | 41.36, 21.10 | 13.57 | 10.83 | 13.14 |
| 6 | 100 | PG, 40 | 125/25, 15 | 4, 45-50 | 41.81, 22.52 | 13.39 | 10.06 | 12.23 |
| 7 | 100 | PG, 20 | 80/20, 17 | 4, 45-50 | 40.75, 21.67 | 14.04 | 11.12 | 12.42 |
| 8 | 50 | ACET, 100 | 40/10, 20 | 24, 20-25 | 43.11, 13.90 | 17.45 | 13.13 | 12.41 |
| 9 | 100 | ACET, 300 | 80/20, 20 | 24, 20-25 | 42.34, 14.99 | 17.25 | 13.09 | 12.33 |
| 10 | 100 | ACET, 400 | 180/20, 10 | 24, 20-25 | 38.66, 18.26 | 17.26 | 13.11 | 12.71 |
| 11 | 100 | ACET, 400 | 180/20, 10 | 24, 20-25 | 40.80, 21.48 | 15.60 | 11.19 | 10.93 |
| 12 | 100 | ACET, 150 | 30/20, 40aq^{c} | 24, 20-25 | 44.58, 13.42 | 18.36 | 13.24 | 10.40 |
| 13 | 100 | ACET, 150 | 30/25, 40aq^{c} | 24, 20-25 | 43.94, 19.63 | 14.25 | 10.39 | 11.79 |
| 14 | 100 | - - -^{d} | 380/20, 5 | 24, 20-25 | 49.21, 15.21 | 13.72 | 10.63 | 11.23 |
| 15 | 100 | - - -^{d} | 380/20, 5 | 24, 20-25 | 46.06, 17.53 | 15.07 | 10.81 | 10.53 |
| 16 | 100 | - - -^{d} | 475/25, 5 | 24, 20-25 | 48.03, 16.02 | 16.02 | 10.44 | 11.26 |
| 17 | 100 | - - -^{d} | 570/30, 5 | 24, 20-25 | 48.33, 15.45 | 14.58 | 10.58 | 11.06 |
| 18 | 100 | - - -^{d} | 225/25, 10 | 24, 20-25 | 44.61, 18.54 | 15.03 | 11.36 | 10.46 |

ªThe saponified mixture also contained approximately 6-8% cucurbitaxanthin and capsanthone as minor carotenoids whose compositions are not shown in Table 1;
^{b}abbreviations: PG, propylene glycol; EtOH, ethanol; ACET, acetone; KOH, potassium hydroxide;
aq^{c}: 40 wt % KOH in water;
^{d}no solvent.

It is imperative to point out that paprika oleoresin also contains 21-23% of minor carotenoids and approximately 10-12% of these carotenoids were identified as cucurbitaxanthin and capsanthone.

However, due to the complexity presented, the work described herein focused on the isolation, separation, and purification of the major carotenoids in paprika oleoresin using the novel processes described herein. The major carotenoids in saponified paprika oleoresin were also accompanied by significant amounts of their cis-isomers; this was particularly noticeable in the case of capsanthin as shown in Table 1. Therefore, the inventor focused on the isolation of the major carotenoids in paprika oleoresin that consisted of trans-isomers of capsanthin, β-carotene, β-cryptoxanthin, and zeaxanthin. This was because it has been well-established that the cis-isomers of carotenoids are not crystallized well due to their increased solubility in nearly all organic solvents. Saponification of carotenoid esters in paprika oleoresin at ambient and low temperature clearly showed that trans-capsanthin accounted for about 39-48% of total carotenoids that was accompanied by about 12-23% of cis-capsanthins that were not expected to crystallize. Consequently, saponification of carotenoid esters in paprika oleoresin at high temperatures can increase the composition of the less desirable cis-carotenoids relative to their trans-counterparts resulting in low recovery of crystallized carotenoids. In addition, saponification at high temperature (60-80° C.) can also result in the degradation of base-sensitive capsanthin to β-citraurin. These disadvantages prompted the inventor to search for an alternative process, where the criteria included identifying processes for low temperature saponification of carotenoid esters in paprika oleoresin.

As shown in Table 1, the relative compositions of the major carotenoids in paprika oleoresin saponified at ambient or low temperature (45-48° C.) were consistent and trans-capsanthin accounted for 39-48% of total carotenoids. As pointed out earlier, the trans-carotenoids in saponified paprika oleoresin were accompanied by significant amounts of their cis-isomers. At the end of saponification, the trans-isomers of the major carotenoids crystallized while their cis-isomers as well as minor carotenoids were removed by filtration. The relative composition of the major crystalline trans-carotenoids that was determined by HPLC after crystallization is shown in Table 2. These crystalline mixtures were subjected to hexane extraction followed by crystallization to increase the purity of the mixture to 80-85% and to separate capsanthin and zeaxanthin from β-carotene and β-cryptoxanthin.

TABLE 2
The relative composition of crystalline mixture of trans-carotenoids after saponification of paprika oleoresin and
removal of cis-isomers and other minor carotenoids before hexane extraction as determined by HPLC.^{a}

| | Paprika | Solvent | EtOH/KOH | Time, Temp. | Capsanthin | β-Carotene | β-Cryptoxanthin | Zeaxanthin |
|---|---|---|---|---|---|---|---|---|
| Exp. | Oleoresin, g | g | g/g, % wt:wt | h, ° C. | % trans, % cis | % | % | % |
| 1 | 50 | PG, 10 | 40/10, 20 | 3, 45-50 | 59.46 | 13.22 | 8.25 | 12.47 |
| 2 | 50 | PG, 10 | 40/10, 20 | 4, 45-50 | 61.86 | 12.71 | 8.62 | 16.81 |
| 3 | 100 | PG, 20 | 80/20, 20 | 4, 45-50 | 63.65 | 13.54 | 7.64 | 15.17 |
| 4 | 100 | PG, 50 | 80/25, 16 | 4, 45-50 | 61.56 | 8.65 | 8.44 | 21.35 |
| 5 | 100 | PG, 50 | 80/25, 16 | 4, 45-50 | 62.36 | 7.38 | 8.70 | 21.56 |
| 6 | 100 | PG, 40 | 125/25, 15 | 4, 45-50 | 60.66 | 11.22 | 4.79 | 23.33 |
| 7 | 100 | PG, 20 | 80/20, 17 | 4, 45-50 | 60.72 | 8.94 | 7.79 | 22.55 |
| 8 | 50 | ACET, 100 | 40/10, 20 | 24, 20-25 | 67.14 | 5.20 | 7.29 | 20.37 |
| 9 | 100 | ACET, 300 | 80/20, 20 | 24, 20-25 | 63.66 | 7.92 | 8.38 | 20.04 |
| 10 | 100 | ACET, 400 | 180/20, 10 | 24, 20-25 | 62.31 | 8.45 | 7.56 | 21.68 |
| 11 | 100 | ACET, 400 | 180/20, 10 | 24, 20-25 | 66.70 | 10.27 | 6.61 | 16.42 |
| 12 | 100 | ACET, 150 | 30/20, 40aq^{b} | 24, 20-25 | 66.33 | 6.07 | 6.97 | 20.63 |
| 13 | 100 | ACET, 150 | 30/25, 40aq^{b} | 24, 20-25 | 66.11 | 2.81 | 5.92 | 25.16 |
| 14 | 100 | - - -^{c} | 380/20, 5 | 24, 20-25 | 65.35 | 11.07 | 7.68 | 15.90 |
| 15 | 100 | - - -^{c} | 380/20, 5 | 24, 20-25 | 64.79 | 11.77 | 7.28 | 16.16 |
| 16 | 100 | - - -^{c} | 475/25, 5 | 24, 20-25 | 64.05 | 10.73 | 8.11 | 17.11 |
| 17 | 100 | - - -^{c} | 570/30, 5 | 24, 20-25 | 65.32 | 10.68 | 7.75 | 16.25 |
| 18 | 100 | - - -^{c} | 225/25, 10 | 24, 20-25 | 63.77 | 9.30 | 7.58 | 19.35 |

^{a}Abbreviations: PG, propylene glycol; EtOH, ethanol; ACET, acetone; KOH, potassium hydroxide;
aq^{b}: 40 wt % KOH in water;
^{c}no solvent.

Further still, the inventor has surprisingly observed that saponification of carotenoid esters in paprika oleoresin can be carried in hexane and ethanol with aqueous solutions of KOH at ambient temperature. Therefore, in certain embodiments, carotenoid esters in paprika oleoresin (100 g & 200 g) were saponified in hexane and ethanol with addition of 40-62% (wt:wt) aqueous solution of KOH in water at ambient temperature after 24 hours. Even though ambient temperature saponification of esters in aqueous solutions is a reversible reaction and does not proceed at ambient temperature, carotenoid esters in paprika oleoresin were successfully saponified by using a highly concentrated solution of KOH in water. This was because upon hydrolysis of carotenoid esters in the presence of hexane, unesterified carotenoids gradually crystallized and shifted the reaction equilibrium forward. Also in these experiments, the aqueous KOH was added to the solution of oleoresin in hexane and ethanol dropwise over a period of 4-5 h to prevent degradation of capsanthin. With this approach, a low concentration of KOH was maintained during saponification that was essential in preserving the integrity of capsanthin. The relative composition of saponified carotenoids in the crude oleoresin determined by HPLC is shown in Table 3.

TABLE 3
The relative composition of major carotenoids after 24 h saponification of carotenoid esters in paprika oleoresin in
hexane and ethanol with aqueous KOH at 20-25° C. before crystallization determined by HPLC.ª

| Exp., | | | | Capsan- | β- | β-Crypto- | | Cucurbita- | Capsan- |
|---|---|---|---|---|---|---|---|---|---|
| Oleoresin | Hexane | EtOH | KOH/H₂O | thin | Carot-ene | xanthin | Zea-xanthin | xanthin | thone |
| g | g | g | g/g, wt. % | % trans, % cis | % | % | % | % | % |
| 19, 100 | 100 | 30 | 25/15, 62 | 39.87, 15.88 | 12.95 | 9.50 | 10.32 | 7.32 | 4.16 |
| 20, 100 | 100 | 30 | 30/18, 62 | 44.73, 12.04 | 11.96 | 8.85 | 9.83 | 7.24 | 5.35 |
| 21, 100 | 75 | 30 | 25/15, 62 | 38.83, 17.77 | 11.61 | 8.91 | 10.59 | 7.25 | 5.04 |
| 22, 100 | 75 | 30 | 30/18, 62 | 42.60, 14.73 | 12.81 | 9.48 | 10.19 | 7.50 | 4.69 |
| 23, 100 | 50 | 30 | 25/15, 62 | 39.67, 15.58 | 13.05 | 9.61 | 10.30 | 7.34 | 4.45 |
| 24, 200 | 200 | 60 | 50/30, 62 | 44.63, 11.64 | 12.09 | 9.12 | 10.19 | 7.12 | 5.21 |
| 25, 200 | 200 | 60 | 50/30, 62 | 45.56, 11.20 | 11.63 | 9.05 | 10.36 | 6.93 | 5.27 |
| 26, 200 | 200 | 60 | 50/30, 62 | 45.34, 13.00 | 11.49 | 8.91 | 9.85 | 5.33 | 6.07 |
| 27, 200 | 200 | 60 | 50/61, 45 | 48.25, 11.42 | 11.87 | 10.15 | 11.92 | 5.93 | 5.56 |
| 28, 200 | 200 | 60 | 50/61, 45 | 46.21, 10.22 | 12.09 | 9.63 | 11.18 | 5.41 | 5.26 |
| 29, 200 | 200 | 80 | 60/90, 40 | 43.40, 12.22 | 13.73 | 9.82 | 10.73 | 5.23 | 4.87 |

ªAbbreviations: EtOH, ethanol; KOH, potassium hydroxide.

The work up of this saponification involved neutralizing the base with a 50% solution of acetic acid in water (v:v) and stirring the products for an additional 24 hours at ambient temperature. This allowed crystallization of trans-capsanthin and trans-zeaxanthin and other minor trans-carotenoids that were simply removed as crystals by filtration and were further purified by washing with water and hexane. This process was shown to be ideal for simultaneous saponification and crystallization of trans-capsanthin and trans-zeaxanthin that were separated from other carotenoids simply by filtration followed by further purification with hexane. The relative composition of crystallized carotenoids after removal of cis-isomers of capsanthin and minor carotenoids is shown in Table 4.

TABLE 4
The relative composition of crystallized trans-carotenoids after 24 h saponification of carotenoid esters in
paprika oleoresin in hexane and ethanol with aqueous KOH at 20-25° C. after removal of cis-isomers
and other minor carotenoids by hexane extraction determined by HPLC.^{a}

| Exp. | | | | | | β-Caro- | β-Crypto- | Cucurbita- | Capsan- |
|---|---|---|---|---|---|---|---|---|---|
| Oleoresin | Hexane | EtOH | KOH/H₂O | Capsanthin | Zeaxanthin | tene | xanthin | xanthin | thone |
| g | g | g | g/g, wt. % | % | % | % | % | % | % |
| 19, 100 | 100 | 30 | 25/15, 62 | 72.13 | 19.32 | 0.00 | 2.98 | 2.78 | 2.79 |
| 20, 100 | 100 | 30 | 30/18, 62 | 75.92 | 17.47 | 0.00 | 2.42 | 2.93 | 1.25 |
| 21, 100 | 75 | 30 | 25/15, 62 | 67.81 | 19.58 | 3.18 | 3.20 | 3.12 | 3.11 |
| 22, 100 | 75 | 30 | 30/18, 62 | 68.62 | 19.30 | 2.26 | 2.95 | 2.74 | 4.13 |
| 23, 100 | 50 | 30 | 25/15, 62 | 64.07 | 17.74 | 6.98 | 4.51 | 2.97 | 3.73 |
| 24, 200 | 200 | 60 | 50/30, 62 | 72.65 | 17.29 | 0.00 | 2.47 | 2.81 | 4.78 |
| 25, 200 | 200 | 60 | 50/30, 62 | 72.04 | 17.55 | 0.00 | 2.57 | 2.40 | 5.44 |
| 26, 200 | 200 | 60 | 50/30, 62 | 71.27 | 15.67 | 0.00 | 6.10 | 2.52 | 4.44 |
| 27, 200 | 200 | 60 | 50/61, 45 | 71.08 | 18.27 | 0.00 | 2.90 | 2.81 | 4.94 |
| 28, 200 | 200 | 60 | 50/61, 45 | 71.66 | 17.81 | 0.00 | 2.60 | 2.80 | 5.13 |
| 29, 200 | 200 | 80 | 60/90, 40 | 70.23 | 18.49 | 0.00 | 2.53 | 4.60 | 4.15 |

^{a}Abbreviations: EtOH, ethanol; KOH, potassium hydroxide.

The saponification of carotenoid esters in experiments 19-23 were carried out with 100 g of paprika oleoresin using a saturated solution of KOH in water (62%) and only 30 g of ethanol that after crystallization afforded a mixture of major and minor trans-carotenoids in 80-85% purity. In experiment 19 and 20, equal weights of hexane and oleoresin (100 g/100 g) were used that resulted in complete removal of β-carotene into the filtrate from crystallization and increased the composition of capsanthin in the mixture. Consequently, the crystallized carotenoids from experiments 19 and 20 contained 72% and 76% of trans-capsanthin, respectively. In experiments 21-23, the weight ratio of hexane/oleoresin was reduced to 75 g/100 g and 50 g/100 g (Table 3) and as a result the crystallized carotenoids contained 2-7% of β-carotene and had lower weight ratio of trans-capsanthin (64-69%). The weight ratio of trans-zeaxanthin in the crystallized product was not affected by the amounts of hexane that was used in saponification since this carotenoid was totally insoluble in hexane. Because the aim of this invention was to obtain a crystalline product that would have the highest relative composition of trans-capsanthin, the scale up saponification experiments were carried out with 200 g of oleoresin and equal weight of hexane (experiments 24-29). When the saponification of carotenoid esters in paprika oleoresin were carried out on 200 g scale with 62% KOH (Exp. 24-26), 45% KOH (Exp. 27 & 28), and 40% KOH (Exp. 29), the relative compositions of the crystallized carotenoids were reproducible and consistent. These saponification experiments afforded 8.70-10.40 g of a mixture of major and minor carotenoids in 80-85% purity that contained 70-73% of trans-capsanthin, 16-18% of trans-zeaxanthin, 2.47-6.10% of β-cryptoxanthin, 2.40-4.60% of cucurbitaxanthin, and 4.15-5.44% of capsanthone. A detailed flow chart of this process for saponification of carotenoid esters in 200 g of paprika oleoresin is shown in FIG. 7.

In the first step, paprika oleoresin was solubilized in hexane and ethanol and an aqueous solution of KOH in water at various concentrations (62%, 45%, or 40%) was added over a period of 4-5 h and the mixture was stirred for about 24 hours at 20-25° C. In the second step, the base was neutralized with a 1:1 solution of acetic acid-water (v:v) and in the third step the mixture was stirred at ambient temperature to promote the crystallization of capsanthin and zeaxanthin. In step four of this process, crystallized carotenoids were filtered and washed with 70° C. water (200 g/200 g of oleoresin) and allowed to dry on the funnel for 4 h. To increase the purity of carotenoids, the solids were then washed with appropriate amounts of hexane in step five of this process. The crystallized carotenoids were then dried under high vacuum at 50° C. that afforded 8.0 g of a mixture of capsanthin (71%), zeaxanthin (18%), β-cryptoxanthin (3%), cucurbitaxanthin (3%), and capsanthone (6%) in 80-85% purity. This crystalline mixture was subjected to extraction with aqueous acetone that afforded 7.0 g of a crystalline mixture of capsanthin (85%) and zeaxanthin (15%) in 90% purity. In an alternatively embodiment, after washing the impure crystalline carotenoids with 70° C. water and drying, the solids can be stirred with hexane for several hours at ambient temperature and filtered to increase the purity of crystallized carotenoids to 80-85%.

The filtrate from this process that contains hexane and ethanol were evaporated under reduced pressure and these solvents were recycled without separation. Hexane (85%), ethanol (12%), and water (3%) form an azeotropic mixture and can be recycled without separation. The presence of small amount of water that is carried over because of azeotropic distillation with ethanol will not present a problem. This is because the saponification of carotenoid esters in paprika oleoresin is carried out with the mixture of these solvents in aqueous KOH. However, the hexane/ethanol ratio will have to be adjusted according to the saponification protocol. The filtrate from saponification also contains excess of acetic acid that does not form an azeotrope with either hexane or ethanol.

### Separation and Purification of Crystalline Mixture of Carotenoids from Saponified Paprika Oleoresin

As shown in FIG. 4, the separation of a crystalline mixture of capsanthin, zeaxanthin, β-carotene, and β-cryptoxanthin obtained from saponification of carotenoid esters in paprika oleoresin was accomplished by sequential extraction with an appropriate solvent. In the first step, β-carotene and β-cryptoxanthin were solubilized and extracted from the mixture of carotenoids with a C₅-C₈ hydrocarbon, preferably hexane while zeaxanthin and capsanthin remained insoluble in hydrocarbon solvents. In a preferred embodiment of the present invention a crystalline mixture (12.53 g) of capsanthin (61.86%), zeaxanthin (16.81%), β-carotene (12.71%), and β-cryptoxanthin (8.62%) was extracted with hexane (350 g) at temperatures in the range of 25-70° C. for 1-3 h. After stirring the mixture at ambient temperature for 3-5 h, the solids were filtered and washed with hexane (60 g). The crystallized solids (9.81 g) consisted of capsanthin (76.36%) and zeaxanthin (23.64%). This crystalline mixture was extracted with aqueous acetone and filtered. After drying the resulting solids, 7.0 g of a mixture of capsanthin (85%) and zeaxanthin (15%) was obtained in 85-90% purity. The filtrate was evaporated to dryness to afford 2.72 g of a mixture of β-carotene (62.99%) and β-cryptoxanthin (37.01%).

In a preferred embodiment, the acetone to water ratio is in the range of 9:1 to 3:1 per gram of the mixture of capsanthin and zeaxanthin.

The following examples are offered to illustrate but not limit the invention. Thus, it is presented with the understanding that various formulation modifications as well as method of delivery modifications may be made and still are within the spirit of the invention.

### Example 1

### Saponification of Carotenoid Esters in Paprika Oleoresin at Low Temperature Using 20% KOH in Ethanol (EtOH) and Propylene Glycol (PG) as Co-Solvent

Paprika oleoresin (50 g) was transferred into a 250 mL round bottom flask equipped with a stir bar and was treated with propylene glycol (PG, 10 g) and a solution of KOH (10 g) in ethanol (40 g) [20% wt:wt]. The flask was placed in an oil bath at 55° C. and the mixture was stirred at 45-50° C. The course of saponification was followed by HPLC that after 3 h that showed complete saponification. Saponification was allowed to proceed for another hour (total of 4 h) and the heat was removed. The mixture was allowed to cool down to ambient temperature and a solution of acetic acid (AcOH) and water (23 mL, 1:1, v:v) was added. Ethanol was evaporated under reduced pressure (100 torr) on a rotary evaporator (bath temperature: 55° C.) at 45-50° C. (solution temperature). Hot water (70° C., 50 g) was added and the mixture was stirred at 70° C. for 1 h until the paste was dissolved and a suspension was obtained. The resulting suspension was filtered while hot. The purple solids were washed with 70° C. water (50 g) and the crystals were dried in an oven at 60° C. under high vacuum for 24 hours to afford a crystalline mixture of carotenoids (4.80 g, 60% total carotenoids, 2.88 g). The relative composition of carotenoids determined by HPLC was: capsanthin (61.86%), β-carotene (12.71%), β-cryptoxanthin (8.62%), and zeaxanthin (16.81%). This crystalline mixture was stirred with hexane (15 g) at ambient temperature for 4 h and filtered. The crystals were washed with hexane (15 g) and dried at 40-60° C. under high vacuum for 24 hours to afford 3.05 g (84% total carotenoids, 2.56 g) of a mixture of capsanthin (76.20%), zeaxanthin (19.18%), and β-cryptoxanthin (4.62%).

### Example 2

### Saponification of Carotenoid Esters in Paprika Oleoresin at Low Temperature Using 20% KOH in Ethanol (EtOH) and Propylene Glycol (PG) as Co-Solvent

Paprika oleoresin (100 g) was transferred into a 500 mL round bottom flask equipped with a stir bar and was treated with propylene glycol (PG, 20 g) and a solution of KOH (20 g) in ethanol (80 g) [20% wt:wt]. The flask was placed in an oil bath at 55° C. and the mixture was stirred at 45-50° C. After 4 h, the heat was removed, the mixture was allowed to cool down to ambient temperature. A solution of acetic acid (AcOH) and water (45 mL, 1:1, v:v) was added and ethanol was evaporated under reduced pressure (100 torr) on a rotary evaporator (bath temperature: 55° C.) at 45-50° C. (solution temperature). Hot water (70° C., 100 g) was added and the mixture was stirred at 70° C. until the paste was dissolved and a suspension was obtained. The mixture was filtered while hot. The purple solids were washed with 70° C. water (100 g) and the crystals were dried in an oven at 60° C. under high vacuum for 24 hours to afford a crystalline mixture of carotenoids (8.30 g, 60% total carotenoids, 4.98 g). The relative composition of carotenoids determined by HPLC was: capsanthin (63.65%), β-carotene (13.54%), β-cryptoxanthin (7.64%), and zeaxanthin (15.17%). This crystalline mixture was stirred with hexane (30 g) at ambient temperature for 4 h and filtered. The crystals were washed with hexane (30 g) and dried at 40-60° C. under high vacuum for 24 hours to afford 5.15 g (85% total carotenoids, 4.37 g) of a mixture of capsanthin (74.5%), zeaxanthin (20.60%), and β-cryptoxanthin (4.90%).

### Example 3

### Saponification of Carotenoid Esters in Paprika Oleoresin at Ambient Temperature Using 20% KOH in Ethanol and Acetone as Co-Solvents

Paprika oleoresin (50 g) was transferred into a 250 mL Erlenmeyer flask equipped with a stir bar and was dissolved in acetone (100 g) and the mixture was treated with a solution of KOH (10 g) in ethanol (40 g) [20% wt:wt] at ambient temperature. This resulted in the formation of significant amounts of soft and hard waxes as well as potassium salts of fatty acids that precipitated out of the solution. The course of saponification was followed by HPLC that showed complete saponification after 24 hours. A solution of acetic acid (AcOH) and water (21 mL, 1:1, v:v) was added and the mixture was stirred at ambient temperature for 20 min. Acetone was first distilled under reduced pressure (300 torr) at 35-40° C. and this was followed by distillation of ethanol at 45-50° C. (100 torr) to afford an oleoresin in water. The oleoresin was treated with hot water (70° C., 50 g) and the mixture was stirred at 70° C. for 1 h until the paste was dissolved and a uniform suspension was obtained. The resulting suspension was filtered while it was hot. The purple solids were washed with 70° C. water (100 g) and the crystals were dried in an oven at 60° C. under high vacuum for 24 hours to afford a crystalline mixture of carotenoids (4.10 g, 62% total carotenoids, 2.54 g). The relative composition of carotenoids determined by HPLC was: capsanthin (67.14%), β-carotene (5.20%), β-cryptoxanthin (7.29%), and zeaxanthin (20.37%). The crystalline mixture was stirred with hexane (15 g) at ambient temperature for 4 h and filtered. The crystals were washed with hexane (15 g) and dried at 40-60° C. under high vacuum for 24 hours to afford 2.76 g (85% total carotenoids, 2.35 g) of a mixture of capsanthin (75.4%), zeaxanthin (21.30%), and β-cryptoxanthin (3.30%).

### Example 4

Saponification of Carotenoid Esters in Paprika Oleoresin at Ambient Temperature Using 20% KOH in Ethanol and Acetone as Co-Solvents Paprika oleoresin (100 g) was transferred into a 500 mL Erlenmeyer flask equipped with a stir bar and was dissolved in acetone (300 g) and the mixture was treated with a solution of KOH (20 g) in ethanol (80 g) [20% wt:wt] at ambient temperature. This resulted in the formation of significant amounts of soft and hard waxes as well as potassium salts of fatty acids that precipitated out of the solution. The course of saponification was followed by HPLC that showed complete saponification after 24 hours. A solution of acetic acid (AcOH) and water (42 mL, 1:1, v:v) was added and the mixture was stirred at ambient temperature for 20 min. Acetone was first distilled under reduced pressure (300 torr) at 35-40° C. and this was followed by distillation of ethanol at 45-50° C. (100 torr) to afford an oleoresin in water. The oleoresin was treated with hot water (70° C., 100 g) and the mixture was stirred at 70° C. for 1 h until a uniform suspension was obtained. The resulting suspension was filtered while it was hot. The purple solids were washed with 70° C. water (200 g) and the crystals were dried in an oven at 60° C. under high vacuum for 24 hours to afford a crystalline mixture of carotenoids (8.50 g, 61% total carotenoids, 5.19 g). The relative composition of carotenoids determined by HPLC was: capsanthin (63.66%), β-carotene (7.92%), β-cryptoxanthin (8.38%), and zeaxanthin (20.04%). The crystalline mixture was stirred with hexane (30 g) at ambient temperature for 4 h and filtered. The crystals were washed with hexane (30 g) and dried at 40-60° C. under high vacuum for 24 hours to afford 5.67 g (83% total carotenoids, 4.71 g) of a mixture of capsanthin (75.8%), zeaxanthin (20.4%), and β-cryptoxanthin (3.8%).

### Example 5

### Saponification of Carotenoid Esters in Paprika Oleoresin in Acetone and Ethanol at Ambient Temperature Using 40% Aqueous Solution of KOH

Paprika oleoresin (100 g) was transferred into a 500 mL Erlenmeyer flask equipped with a stir bar and was dissolved in acetone (150 g) and ethanol (30 g) and the mixture was treated dropwise with a solution of KOH (20 g) in water (30 g) [40% wt:wt] in 4 h at ambient temperature. The mixture was stirred at ambient temperature for 24 hours. A solution of acetic acid (AcOH) and water (61 mL, 1:1, v:v) was added and the mixture was stirred at ambient temperature for 2 h. Acetone was first distilled under reduced pressure (300 torr) at 35-40° C. and this was followed by distillation of ethanol at 45-50° C. (100 torr) to afford an oleoresin in water. The oleoresin was treated with hot water (70° C., 100 g) and the mixture was stirred at 70° C. for 1 h until a uniform suspension was obtained. The resulting suspension was filtered while it was hot. The purple solids were washed with 70° C. water (200 g) and the crystals were dried in an oven at 60° C. under high vacuum for 24 hours to afford a crystalline mixture of carotenoids (8.20 g, 62% total carotenoids, 5.08 g). The relative composition of carotenoids determined by HPLC was: capsanthin (66.33%), β-carotene (6.07%), β-cryptoxanthin (6.97%), and zeaxanthin (20.63%). The crystalline mixture was stirred with hexane (30 g) at ambient temperature for 4 h and filtered. The crystals were washed with hexane (30 g) and dried at 40-60° C. under high vacuum for 24 hours to afford 5.60 g (85% total carotenoids, 4.76 g) of a mixture of capsanthin (74.4%), zeaxanthin (20.8%), and β-cryptoxanthin (4.8%).

### Example 6

### Saponification of Carotenoid Esters in Paprika Oleoresin at Ambient Temperature Using 5% KOH in Ethanol

Paprika oleoresin (100 g) was transferred into a 1000 mL Erlenmeyer flask equipped with a stir bar and was treated with a solution of KOH (20 g) in ethanol (380 g) [5% wt:wt] at ambient temperature. The course of saponification was followed by HPLC that showed complete saponification after 24 hours. The saponified oleoresin was treated with 42 mL of an aqueous solution of acetic acid-water (v:v) and the mixture was stirred at ambient temperature for 30 min. Ethanol was evaporated under reduced pressure 45-50° C. (100 torr) to afford a red paste. The oleoresin was treated with hot water (70° C., 100 g) and the mixture was stirred at 70° C. until the paste was dissolved and a suspension was obtained. The resulting suspension was filtered while it was hot. The purple solids were washed with 70° C. water (200 g) and the crystals were dried in an oven at 60° C. under high vacuum for 24 hours to afford a crystalline mixture of carotenoids (9.00 g, 65% total carotenoids). The relative composition of carotenoids determined by HPLC was: capsanthin (65.35%), β-carotene (11.07%), β-cryptoxanthin (7.68%), and zeaxanthin (15.90%).

### Example 7

Saponification of Carotenoid Esters in Paprika Oleoresin at Ambient Temperature Using 5% KOH in Ethanol

Paprika oleoresin (100 g) was transferred into a 1000 mL Erlenmeyer flask equipped with a stir bar and was treated with a solution of KOH (20 g) in ethanol (380 g) [5% wt:wt] at ambient temperature. The course of saponification was followed by HPLC that showed complete saponification after 24 hours. The saponified oleoresin was treated with 42 mL of an aqueous solution of acetic acid-water (v:v) and the mixture was stirred at ambient temperature for 30 min. Ethanol was evaporated under reduced pressure 45-50° C. (100 torr) to afford a red paste. The oleoresin was treated with hot water (70° C., 100 g) and the mixture was stirred at 70° C. until the paste was dissolved and a suspension was obtained. The resulting suspension was filtered while it was hot. The purple solids were washed with 70° C. water (200 g) and the crystals were dried in an oven at 60° C. under high vacuum for 24 hours to afford a crystalline mixture of carotenoids (9.35 g, 63% total carotenoids). The relative composition of carotenoids determined by HPLC was: capsanthin (64.79%), β-carotene (11.77%), β-cryptoxanthin (7.28%), and zeaxanthin (16.16%).

### Example 8

### Saponification of Carotenoid Esters in Paprika Oleoresin at Ambient Temperature Using 5% KOH in Ethanol

Paprika oleoresin (100 g) was transferred into a 1000 mL Erlenmeyer flask equipped with a stir bar and was treated with a solution of KOH (25 g) in ethanol (475 g) [5% wt:wt] at ambient temperature. The course of saponification was followed by HPLC that showed complete saponification after 24 hours. The saponified oleoresin was treated with 51 mL of an aqueous solution of acetic acid-water (v:v) and the mixture was stirred at ambient temperature for 30 min. Ethanol was evaporated under reduced pressure 45-50° C. (100 torr) to afford a red paste. The oleoresin was treated with hot water (70° C., 100 g) and the mixture was stirred at 70° C. until the paste was dissolved and a suspension was obtained. The resulting suspension was filtered while it was hot. The purple solids were washed with 70° C. water (200 g) and the crystals were dried in an oven at 60° C. under high vacuum for 24 hours to afford a crystalline mixture of carotenoids (9.72 g, 64% total carotenoids). The relative composition of carotenoids determined by HPLC was: capsanthin (64.05%), β-carotene (10.73%), β-cryptoxanthin (8.11%), and zeaxanthin (17.11%).

### Example 9

### Saponification of Carotenoid Esters in Paprika Oleoresin at Ambient Temperature Using 5% KOH in Ethanol

Paprika oleoresin (100 g) was transferred into a 1000 mL Erlenmeyer flask equipped with a stir bar and was treated with a solution of KOH (30 g) in ethanol (570 g) [5% wt:wt] at ambient temperature. The course of saponification was followed by HPLC that showed complete saponification after 24 hours. The saponified oleoresin was treated with 61 mL of an aqueous solution of acetic acid-water (v:v) and the mixture was stirred at ambient temperature for 30 min. Ethanol was evaporated under reduced pressure 45-50° C. (100 torr) to afford a red paste. The oleoresin was treated with hot water (70° C., 100 g) and the mixture was stirred at 70° C. until the paste was dissolved and a suspension was obtained. The resulting suspension was filtered while it was hot. The purple solids were washed with 70° C. water (200 g) and the crystals were dried in an oven at 60° C. under high vacuum for 24 hours to afford a crystalline mixture of carotenoids (8.47 g, 60% total carotenoids). The relative composition of carotenoids determined by HPLC was: capsanthin (65.32%), β-carotene (10.68%), β-cryptoxanthin (7.75%), and zeaxanthin (16.25%).

### Example 10

### Saponification of Carotenoid Esters in Paprika Oleoresin in Hexane and Ethanol at Ambient Temperature Using 62% Aqueous Solution of KOH

Paprika oleoresin (200 g) was transferred into a 1000 mL Erlenmeyer flask equipped with a stir bar and was stirred in hexane (200 g) and ethanol (60 g) at ambient temperature until the oleoresin dissolved. A saturated solution of KOH (50 g) in water (30 g) [62% wt:wt] was transferred into an addition funnel and was added dropwise to the oleoresin in hexane and ethanol in 4 h at 20-25° C. The saponification generated heat and this slow addition allowed the saponification to proceed at ambient temperature. After 24 hours, a 1:1 solution of AcOH—H₂O (152 mL, v:v) was added dropwise over a period of 1 h; the slow addition of acid to base was essential to maintain the temperature of the mixture below 30° C. The relative composition of carotenoids in the crude saponified mixture was: trans-capsanthin (44.63%), cis-capsanthins (11.64%), β-carotene (12.09%), β-cryptoxanthin (9.12%), zeaxanthin (10.19%), cucurbitaxanthin (7.12%), and capsanthone (5.21%). The mixture was stirred at ambient temperature for 24 hours to allow crystallization of trans-capsanthin in hexane. The saponified oleoresin was filtered and the filtrate was saved for solvent evaporation and recovery. The solids were then washed with hot water (70° C., 200 g) and the crystals were allowed to dry on the funnel for 4 h. The crystals were washed with hexane (60 g) and allowed to dry on the funnel for 1 h. The crystals were removed and dried in a vacuum oven at 50° C. for 24 hours to afford a mixture of trans-capsanthin (72.65%), zeaxanthin (17.29%), β-cryptoxanthin (2.47%), cucurbitaxanthin (2.81%), and capsanthone (4.78%) (8.41 g, 85% pure, 7.14 g). The saved filtrate and the hexane wash were combined, and hexane and ethanol were evaporated and recovered under reduced pressure (230 torr) to 45° C. (100 torr) at 40° C.

### Example 11

### Saponification of Carotenoid Esters in Paprika Oleoresin in Hexane and Ethanol at Ambient Temperature Using 45% Aqueous Solution of KOH

Paprika oleoresin (200 g) was transferred into a 1000 mL Erlenmeyer flask equipped with a stir bar and was stirred in hexane (200 g) and ethanol (60 g) at ambient temperature until the oleoresin dissolved. A saturated solution of KOH (50 g) in water (61 g) [45% wt:wt] was transferred into an addition funnel and was added dropwise to the oleoresin in hexane and ethanol in 4 h at 20-25° C. The saponification generated heat and this slow addition allowed the saponification to proceed at ambient temperature. After 24 hours, a 1:1 solution of AcOH—H₂O (152 mL, v:v) was added dropwise over a period of 1 h; the slow addition of acid to base was essential to maintain the temperature of the mixture below 30° C. The relative composition of carotenoids in the crude saponified mixture was: trans-capsanthin (48.25%), cis-capsanthins (11.42%), β-carotene (11.87%), β-cryptoxanthin (10.15%), zeaxanthin (11.92%), cucurbitaxanthin (5.93%), and capsanthone (5.56%). The mixture was stirred at ambient temperature for 24 hours to allow crystallization of trans-capsanthin in hexane. The saponified oleoresin was filtered and the filtrate was saved for solvent evaporation and recovery. The solids were then washed with hot water (70° C., 200 g) and the crystals were allowed to dry on the funnel for 4 h. The crystals were washed with hexane (60 g) and allowed to dry on the funnel for 1 h. The crystals were removed and dried in a vacuum oven at 50° C. for 24 hours to afford a mixture of trans-capsanthin (71.08%), zeaxanthin (18.27%), β-cryptoxanthin (2.90%), cucurbitaxanthin (2.81%), and capsanthone (4.94%) (9.28 g, 84% pure, 7.80 g). The saved filtrate and the hexane wash were combined, and hexane and ethanol were evaporated and recovered under reduced pressure (230 torr) to 45° C. (100 torr) at 40° C.

### Example 12

### Saponification of Carotenoid Esters in Paprika Oleoresin in Hexane and Ethanol at Ambient Temperature Using 40% Aqueous Solution of KOH

Paprika oleoresin (200 g) was transferred into a 1000 mL Erlenmeyer flask equipped with a stir bar and was stirred in hexane (200 g) and ethanol (60 g) at ambient temperature until the oleoresin dissolved. A saturated solution of KOH (60 g) in water (90 g) [40% wt:wt] was transferred into an addition funnel and was added dropwise to the oleoresin in hexane and ethanol in 4 h at 20-25° C. The saponification generated heat and this slow addition allowed the saponification to proceed at ambient temperature. After 24 hours, a 1:1 solution of AcOH—H₂O (152 mL, v:v) was added dropwise over a period of 1 h; the slow addition of acid to base was essential to maintain the temperature of the mixture below 30° C. The relative composition of carotenoids in the crude saponified mixture was: trans-capsanthin (43.40%), cis-capsanthins (12.22%), β-carotene (13.73%), β-cryptoxanthin (9.82%), zeaxanthin (10.73%), cucurbitaxanthin (5.23%), and capsanthone (4.87%). The mixture was stirred at ambient temperature for 24 hours to allow crystallization of trans-capsanthin in hexane. The saponified oleoresin was filtered and the filtrate was saved for solvent evaporation and recovery. The solids were then washed with hot water (70° C., 200 g) and the crystals were allowed to dry on the funnel for 4 h. The crystals were washed with hexane (60 g) and allowed to dry on the funnel for about 1 hour. In certain embodiments, the drying may occur over one or more hours, for instance between about 1 to 24 hours. The crystals were removed and dried in a vacuum oven at 50° C. for 24 hours to afford a mixture of trans-capsanthin (70.23%), zeaxanthin (18.49%), β-cryptoxanthin (2.53%), cucurbitaxanthin (4.60%), and capsanthone (4.15%) (9.64 g, 83% pure, 7.99 g). The saved filtrate and the hexane wash were combined, and hexane and ethanol were evaporated and recovered under reduced pressure (230 torr) to 45° C. (100 torr) at 40° C.

It should be appreciated that minor modification to the concentrations of reagent, saponification conditions, and conditions for separation of carotenoids, their composition and the ranges expressed herein may be made and still come within the scope and spirit of the present invention.

Having described the invention with reference to particular compositions, theories of effectiveness, and the like, it will be apparent to those of skilled in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

It should be further appreciated that minor dosage and formulation modifications of the composition and the ranges expressed herein may be made and still come within the scope and spirit of the present invention.

It is also to be understood that the formulations and processes described in the specification are simply exemplary embodiments of the inventive concepts defined in the appended claims. Hence, specific conditions and other physical characteristics relating to the embodiments disclosed herein are not to be considered as limiting, unless the claims expressly state otherwise. Where a range of values is provided, it is understood that each intervening value, to the tenth of the unit of the lower limit unless the context clearly dictates otherwise, between the upper and lower limit of that range, and any other stated or intervening value in that stated range, is encompassed within the scope of the present disclosure. The upper and lower limits of these smaller ranges may independently be included in the smaller ranges, and are also encompassed within the scope of the present disclosure, subject to any specifically excluded limit in the stated range. Where the stated range includes one or both of the limits, ranges excluding either or both of those included limits are also included in the scope of the present disclosure. All ranges and parameters, including but not limited to percentages, parts, and ratios, disclosed herein are understood to encompass any and all sub-ranges assumed and subsumed therein, and every number between the endpoints. For example, a stated range of “1 to 10” should be considered to include any and all sub-ranges beginning with a minimum value of 1 or more and ending with a maximum value of 10 or less (e.g., 1 to 6.1, or 2.3 to 9.4), and to each integer (1, 2, 3, 4, 5, 6, 7, 8, 9, 10) contained within the range. Except in certain examples or where expressly disclosed herein, all numerical values in this specification and the appended claims are understood to be modified by the word “about” when describing the scope of the numerical range.

In this specification and the appended claims, the singular forms “a,” “an” and “the” include plural reference unless the context clearly dictates otherwise. All combinations of method steps or process steps as used herein can be performed in any order, unless otherwise specified or clearly implied to the contrary by the context in which the referenced combination is made

To the extent that the terms “includes” or “including” or “have” or “having” are used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A” or “B” or both “A” and “B”. When the Applicant intends to indicate “only A or B but not both” then the term “only A or B but not both” or similar structure will be employed. Thus, use of the term “or” herein is the inclusive, and not the exclusive use. Also, to the extent that the terms “in” or “into” are used in the specification or the claims, it is intended to additionally mean “on” or “onto.” In this specification and the appended claims, the singular forms “a,” “an” and “the” include plural reference unless the context clearly dictates otherwise.

The foregoing description has been presented for the purposes of illustration and description. It is not intended to be an exhaustive list or limit the invention to the precise forms disclosed. It is contemplated that other alternative processes and methods obvious to those skilled in the art are considered included in the invention. The description is merely examples of embodiments. It is understood that any other modifications, substitutions, and/or additions may be made, which are within the intended spirit and scope of the disclosure. From the foregoing, it can be seen that the exemplary aspects of the disclosure accomplish at least all of the intended objectives.

The foregoing description has been presented for the purposes of illustration and description. It is not intended to be an exhaustive list or limit the invention to the precise forms disclosed. It should be appreciated that in some exemplary embodiments, well-known processes, well-known methods, devices, and technologies are not described in detail. Persons of ordinary skill in the art will understand that modifications and variations of the embodiments disclosed can be made within the scope of the present invention in order to achieve substantially similar results.

### REFERENCES

1. Deli J., Molnar P. Paprika Carotenoids: Analysis, Isolation, Structure Elucidation, Current Organic chemistry. 2002, 6(13), 1197-1219.
2. Maoka T., Fujiwara Y., Hashimoto K., Akimoto N. Isolation of a Series of Apocarotenoids from the Fruits of the Red Paprika (Capsicum annuum L.). J. Agric. Food Chem. 2001, 49, 1601-1606.
3. Matsufuji H., Nakamura H., Chino M.; Takeda M. Antioxidant Activity of Capsanthin and the Fatty Acid Esters in Paprika (Capsicum annuum). J. Agric. Food Chem. 1998, 46(9), 3468-3472.
4. Hernandez-Ortega M., Ortiz-Moreno A., Hernandez-Navarro M.D., Chamorro-Cevallos G., Dorantes-Alvarez L., Necoechea-Mondragon H. Antioxidant, antinociceptive, and anti-inflammatory effects of carotenoids extracted from dried pepper (Capsicum annuum L.). J. Biomed. and Biotech. 2012, 524019, 10 pp.
5. Narisawa T. Fukaura Y, Hasebe M., Nomura S., Oshima S., Inakuma T. Prevention of N-methylnitrosourea-induced colon carcinogenesis in rats by oxygenated carotenoid capsanthin and capsanthin-rich paprika juice. Proceedings of the Society for Experimental Biology and Medicine. 2000, 224(2), 116-122.
6. Maoka T., Mochida K., Kozuka M., Ito Y., Fujiwara Y., Hashimoto K., Enjo F., Ogata M., Nobukuni Y., Tokuda H. et al. Cancer chemopreventive activity of carotenoids in the fruits of red paprika Capsicum annuum L. Cancer Letters. 2001, 172(2), 103-109].
7. Jo S. J., Kim J. W., Choi H. O., Kim J. H., Kim-J., Woo S. H., Han B. H. Capsanthin inhibits both adipogenesis in 3T3-L1 preadipocytes and weight gain in high-fat diet-induced obese mice. Biomolecules & Therapeutics. 2017, 25(3), 329-336.
8. Aizawa K., Inakuma T. Dietary capsanthin, the main carotenoid in paprika (Capsicum annuum), alters plasma high-density lipoprotein-cholesterol levels and hepatic gene expression in rats British J. of Nutrition. 2009, 102(12), 1760-1766.
9. Joo H. K., Lee Y. R., Lee E. O., Kim S., Jin H., Kim S., Lim Y. P., An C. G, Jeon B. H. Protective Role of Dietary Capsanthin in a Mouse Model of Nonalcoholic Fatty Liver Disease. J. Medicinal Food. 2021, 24(6), 635-644.
10. Fernandez-Garcia E. Carvajal-Lerida I., Perez-Galvez A. Carotenoids exclusively synthesized in red pepper (capsanthin and capsorubin) protect human dermal fibroblasts against UVB induced DNA damage. Photochem. & Photobiologic. Sci. 2016, 15(9), 1204-1211.
11. Maeda H., Nishino A., Maoka T. Biological Activities of Paprika Carotenoids, Capsanthin and Capsorubin. Advances in Experimental Medicine & Biology. 2021, 1261, 285-293.
12. Duanmu et al. China, CN101906254 A 2010-12-08, Method for extraction of capsanthin ointment from dry paprika by using dimethyl ether as solvent.
13. Reilly et a. US20110282083 A1 2011-11-17. Process of converting esterified xanthophylls from Capsicum to non-esterified xanthophylls in high yields and purities.
14. Jacob et al. WO2011135519 A1 2011-11-03, Extraction of capsanthin-rich carotenoid mixture from Capsicum annuum oleoresin.
15. Umigai et al., JP2015174858 A 2015-10-05. Capsicum annuum (red pepper) pigment extract containing β-cryptoxanthin and capsanthin at controlled ratio.
16. Sunilkumar et al. U.S. Pat. No. 9,771,323 B2, Sep. 26, 2017, Omni Active Health Technology Ltd. beta-Cryptoxanthin from plant source and process for its preparation.
17. Sunilkumar et al. U.S. Pat. No. 10,301,259 B2, May 28, 2019, Omni Active Health Technology Ltd. beta-Cryptoxanthin from plant source and process for its preparation.
18. Deshpande et al. U.S. Pat. No. 10,568,846 B2, Feb. 25, 2020, beta-Cryptoxanthin compositions, processes for preparation and uses thereof.
19. Mehta S. US 2022/0009886 A1, Jan. 18, 2022, Manufacture of carotenoid compositions
20. Zechmeister Cholnoky L. The studies on paprika dye; citraurin from capsanthin. Justus Liebigs Annalen der Chemie, 1937, 530, 291-300.
21. Cholnoky L., Szabolcs J. The structure of cryptocapsin, Tetrahedron Letters, 1963, No. 19, 1257-1259.

1. A process for low temperature saponification of carotenoid esters in paprika oleoresin to afford a mixture of capsanthin, β-carotene, β-cryptoxanthin, and zeaxanthin comprising:
treating paprika oleoresin with a non-aqueous solution of potassium hydroxide (KOH) or sodium hydroxide or other alkali metal hydroxides in ethanol (EtOH) or other C1-C3 alcohols and propylene glycol (PG) at a low temperature of about 45 to 50° C. to obtain a saponification mixture;
heating the mixture to a temperature ranging from about 40 to about 50° C. in order to saponify carotenoid esters;
treating the saponified paste with water and a 1:1 solution of acetic acid (AcOH) or other weak organic acids such as propanoic acid, butyric acid in water (v:v) to neutralize the base;
distilling and recovering ethanol under reduced pressure, for instance about 200-120 torr, at about 45 to 50° C. to obtain a saponified oleoresin;
treating the saponified oleoresin with water to obtain a suspension of carotenoids;
filtering the suspension and washing the crystals to obtain a crystalline mixture of trans-capsanthin, trans-β-carotene, trans-β-cryptoxanthin, and trans-zeaxanthin; and
drying the crystalline mixture.

2. The process of claim 1, wherein the weight ratio of paprika oleoresin:PG:EtOH:KOH is in the range of 5:1:4:1 to 5:2:8:2.

3. The process of claim 2, wherein the concentration of KOH in EtOH is in the range of about 10 to 30% by weight.

4. The process of claim 1, wherein the composition of crystalline mixture of carotenoids is about 50-70% by weight trans-capsanthin, about 10-15% by weight trans-β-carotene, about 10-15% by weight trans-β-cryptoxanthin, and about 10-25% by weight trans-zeaxanthin.

5. The process of claim 1, wherein the crystalline mixture of trans-capsanthin, β-carotene, β-cryptoxanthin, and zeaxanthin are separated by extraction with a C5-C₇ hydrocarbon; preferably hexane, to obtain a hydrocarbon soluble fraction and insoluble crystals.

6. The process of claim 5, wherein the hydrocarbon soluble fraction consists of β-carotene and β-cryptoxanthin and the crystals consist of trans-capsanthin and trans-zeaxanthin.

7. The process of claim 6, wherein the composition ratio of crystalline trans-capsanthin to trans-zeaxanthin is in the range of 95%5% to 60%:40%.

8. The process of claim 6, wherein the hydrocarbon soluble fraction is evaporated to afford a crystalline mixture of trans-β-carotene and trans-β-cryptoxanthin.

9. The process of claim 8, wherein the relative composition of crystalline trans-β-carotene:trans-β-cryptoxanthin is in the range of 60%:40% to 20%:80%.

10. The process of claim 7, wherein trans-capsanthin and trans-zeaxanthin are separated from an aqueous solution of acetone in which trans-capsanthin is soluble and trans-zeaxanthin remains as crystals and is removed by filtration.

11. The process of claim 10, wherein trans-capsanthin is crystallized from water by distillation of acetone.

12. The process of claim 11, wherein crystalline trans-capsanthin is dried under high vacuum and results in about 60 to 90% purity.

13. The process of claim 8, wherein trans-β-carotene and trans-β-cryptoxanthin are separated by extraction with a C₁-C₃ alcohols; preferably ethanol, to obtain an alcohol soluble fraction and insoluble crystals.

14. The process of claim 13, wherein the alcohol insoluble fraction consists of trans-β-carotene that is removed by filtration.

15. The process of claim 13, wherein the alcohol soluble fraction consists of trans-β-cryptoxanthin that is obtained by evaporation of alcohol as crystals in 60-75% purity.

16. A process for ambient temperature saponification of carotenoid esters in paprika oleoresin to afford a mixture of capsanthin, β-carotene, β-cryptoxanthin, and zeaxanthin comprising:
solubilizing paprika oleoresin in acetone and treating the solution with a non-aqueous solution of potassium hydroxide (KOH) in ethanol (EtOH) at ambient temperature or aqueous solution of KOH (40-45%) with stirring to saponify carotenoid esters and obtain a saponification mixture;
treating the saponified mixture with a 1/1 solution of acetic acid (AcOH) in water (v:v) to neutralize the base to obtain a suspension of carotenoids;
distilling and recovering acetone and ethanol from the filtrate to obtain a saponifiedoleoresin;
adding water and filtering the suspension and washing the crystals with water to obtain a crystalline mixture of trans-capsanthin, trans-β-carotene, trans-β-cryptoxanthin, and trans-zeaxanthin; and
drying the crystalline mixture of forementioned carotenoids.

17. The process of claim 16, wherein the weight ratio of paprika oleoresin:acetone:EtOH:KOH is in the range of 5:15:4:1 to 5:20:9:1.

18. The process of claim 17, wherein the concentration of KOH in EtOH is in the range of about 10 to 30%.

19. The process of claim 16, wherein the weight ratio of paprika oleoresin:acetone:EtOH:aqueous KOH is in the range of 4:6:1:1 to 5:8:2:1.

20. The process of claim 19, wherein the concentration of KOH in water by weight is in the range of about 40 to 45%.

21. The process of claim 16, wherein the composition of crystalline mixture of carotenoids is about 50-70% by weight trans-capsanthin, about 10-15% by weight trans-β-carotene, about 5-10% by weight trans-β-cryptoxanthin, and about 10-20% by weight trans-zeaxanthin.

22. The process of claim 16, wherein the crystalline mixture of trans-capsanthin, trans-β-carotene, trans-β-cryptoxanthin, and trans-zeaxanthin are each separated in order to obtain purity in the range of about 50%-90%.

23. A process for ambient temperature saponification of carotenoid esters in paprika oleoresin to afford a mixture of capsanthin, β-carotene, β-cryptoxanthin, and zeaxanthin comprising:
treating paprika oleoresin with a non-aqueous solution of potassium hydroxide (KOH) in ethanol (EtOH) at ambient temperature to obtain a saponification mixture;
stirring the saponification mixture at ambient temperatures for about 6 to 24 hours to saponify carotenoid esters;
treating the saponified oleoresin with a 1:1 solution of acetic acid (AcOH)-water (v:v);
distilling and recovering ethanol from the mixture under reduced pressure at 45-50° C. to obtain a saponified paste;
treating the paste with hot water;
filtering the suspension and washing the crystals to obtain a crystalline mixture of trans-capsanthin, trans-β-carotene, trans-β-cryptoxanthin, and trans-zeaxanthin; and
drying the crystalline mixture.

24. The process of claim 21, wherein the weight ratio of ethanol:paprika oleoresin is in the range of 3:1 to 6:1 and the weight ratio of oleoresin to KOH is in the range of 3.3:1 to 5:1.

25. The process of claim 22, wherein the concentration of KOH in EtOH is about 5-10%.

26. The process of claim 21, wherein the composition of crystalline mixture of carotenoids comprises trans-capsanthin in an amount ranging from about 50 to 70% by weight, trans-β-carotene in an amount ranging from about 10 to 15% by weight, trans-β-cryptoxanthin in an amount ranging from about 5 to 10% by weight, and trans-zeaxanthin in an amount ranging from about 10 to 20% by weight.

27. The process of claim 21, wherein the crystalline mixture of trans-capsanthin, trans-β-carotene, trans-β-cryptoxanthin, and trans-zeaxanthin are each separated in high purity according to the process described in claims 5-15.

28. A process for ambient temperature saponification of carotenoid esters in paprika oleoresin to afford a mixture of capsanthin, β-carotene, β-cryptoxanthin, zeaxanthin, cucurbitaxanthin, and capsanthone comprising:
treating a solution of paprika oleoresin in hexane and ethanol with an aqueous solution of potassium hydroxide (KOH) in water over a period of about 4 to 5 hours at ambient temperature to obtain a saponification mixture;
stirring the saponification mixture for at least 24 hours to saponify carotenoid esters;
treating the saponified mixture with a 1:1 solution of acetic acid (AcOH):water (v:v) and stirring the mixture for 24 hours at ambient temperature to crystallize trans-capsanthin and trans-zeaxanthin as major carotenoids and β-carotene, β-cryptoxanthin, cucurbitaxanthin, and capsanthone as minor carotenoids;
filtering the crystallized carotenoids and washing the crystals with hot water at a temperature of about 50-70° C.;
and allowing the crystals to dry for more than one hour;
washing the crystals with a hydrocarbon solvent, preferably hexane to increase the purity of carotenoids; and
drying the crystals at about 45 to 50° C. under high vacuum to obtain a crystalline mixture of trans-capsanthin and trans-zeaxanthin as major carotenoids and β-carotene, β-cryptoxanthin, cucurbitaxanthin, and capsanthone as minor carotenoids in 80% or more purity.

29. The process of claim 28 wherein the weight ratio of oleoresin:hexane:ethanol:KOH is in the range of 3.3:2:1:1 to 4:4:1.2:1.

30. The process of claim 28 wherein the concentration of KOH in water is in the range of about 40 to 62% by weight.

31. The process of claim 28 wherein capsanthin, the major carotenoid in paprika oleoresin is crystallized during ambient saponification and is protected from degradation by KOH in alcohol.

32. The process of claim 28 wherein the composition of crystalline mixture of carotenoids contains trans-capsanthin in an amount ranging from about 60 to 80% by weight, trans-zeaxanthin in an amount ranging from about 10 to 20% by weight, β-carotene in an amount ranging from 0 to about 4% by weight, β-cryptoxanthin in an amount ranging from about 2 to 10% by weight, cucurbitaxanthin in an amount ranging from about 2 to 5% by weight, and capsanthone in an amount ranging from about 1 to 5% by weight, wherein the carotenoids in the crystalline mixture have a purity of 80% or more.

33. The process of claim 28 wherein the filtrate from saponification is enriched in β-carotene and β-cryptoxanthin.

34. The process of claim 28 wherein the major carotenoids capsanthin and zeaxanthin are separated and further purified from minor carotenoids β-carotene, β-cryptoxanthin, cucurbitaxanthin, capsanthone.

35. The process of claim 28 wherein the crystalline mixture comprises trans-capsanthin in an amount ranging from about 60 to 80% by weight, trans-zeaxanthin in an amount ranging from about 10-20% by weight, β-carotene in an amount ranging from about 0 to 4%, β-cryptoxanthin in an amount ranging from about 2 to 10% by weight, cucurbitaxanthin in an amount ranging from about 2 to 5% by weight, capsanthone in an amount ranging from about 1 to 5% by weight is extracted with aqueous acetone to obtain a crystalline mixture of about 85% trans-capsanthin and about 15% zeaxanthin in 80% or more purity.

36. The process of claim 35 wherein the ratio of acetone to water is 9:1 to 3:1 per gram of mixture of capsanthin and zeaxanthin.
